(19) United States
(12) Patent Application Publication
(10) Pub. No.: US 2013/0331894 A1
(43) Pub. Date: Dec. 12, 2013

(54) Bone Screw Structure for a Minimally Invasive Surgery

(71) Applicant: Chih-Hsuan Wei, Chiayi City (TW)

(72) Inventor: Chih-Hsuan Wei, Chiayi City (TW)

(21) Appl. No.: 13/491,689

(22) Filed: Jun. 8, 2012

(51) Int. Cl. A61B 17/86

(52) U.S. Cl. 606/308

(57) ABSTRACT

A bone screw structure for a minimally invasive surgery contains: a body including a holder and an axial seat, the holder having a through hole and two first peripheral walls, each having first threads; the screwing nail being axially connected with the axial seat of the body and having an inner orifice. The each first peripheral wall of the holder including a second side face extending therefrom, between the each first peripheral wall and the second side face is defined a V-shaped slot, and the second side face has second threads. Thereby, the two first peripheral walls of the holder of the body include two second side faces extending therefrom, and between the two first peripheral wall and the two second side faces are defined two V-shaped slots to provide an easy operating space to the bone screw and to lower an area of an opening of a wound.

### BACKGROUND OF THE INVENTION

1. Field of the Invention

The present invention relates to a bone screw structure for a minimally invasive surgery in which two first peripheral walls of a holder of a body include two second side faces extending therefrom, and between the two first peripheral wall and the two second side faces are defined two V-shaped slots to provide an easy operating space to a bone screw and to lower an area of an opening of a wound.

2. Description of the Prior Art

A conventional bone screw structure is short that can not extend out of a patent's body, so as implanting the bone screw structure in a surgery, an opening with a large area is cut to form a large wound to operate the bone screw.

As illustrated in FIG. 4, a conventional bone screw structure comprises a body a0, the body a0 includes a holder al and an axial seat a2, the holder al has a through hole all and two first peripheral walls a12, and each first peripheral wall a12 has first threads a121.

A locking nail b0 is axially connected with the axial seat a2 of the body a0 and includes an inner orifice b1 (not shown), the locking nail b0 further includes a plurality of apertures b2 communicating with the inner orifice b1.

However, the conventional bone screw structure still has the following defects:

1. The conventional bone screw structure is short that can not extend out of a patient's body.

2. The conventional bone screw structure will cause an opening of a wound in a large size, so that the wound recovers slowly.

3. The conventional bone screw structure will increase the area of the opening of the wound to enhance an infection risk.

4. The conventional bone screw structure will break the patient's organization in a surgery.

Although the conventional bone screw structure can screw and fix a spine, it increases the area of the opening of the wound, thus enhancing an infection risk and prolonging a recovery of the wound.

The present invention has arisen to mitigate and/or obviate the afore-described disadvantages.

### SUMMARY OF THE INVENTION

The primary object of the present invention is to provide a bone screw structure for a minimally invasive surgery which is capable of overcoming the shortcomings of the conventional bone screw structure.

Another object of the present invention is to provide a bone screw structure for a minimally invasive surgery which provides an easy operating space to a bone screw and lowers an area of an opening of a wound.

A bone screw structure for a minimally invasive surgery in accordance with a preferred embodiment of the present invention contains:

a body including a holder and an axial seat, the holder having a through hole and two first peripheral walls, and each first peripheral wall having first threads;

the screwing nail being axially connected with the axial seat of the body and having an inner orifice;

characterized in that:

the each first peripheral wall of the holder including a second side face extending therefrom, between the each first peripheral wall and the second side face is defined a V-shaped slot, and the second side face has second threads;

wherein the first threads and the second threads have an identical rotating direction and are formed successively.

wherein the screwing nail includes a plurality of apertures communicating with the inner orifice.

Thereby, the features and advantages of the bone screw structure for the minimally invasive surgery:

1. The bone screw structure is designed in a long structure to extend out of a patient's body.

2. The bone screw structure provides an easy operating space to a bone screw and lowers an area of an opening of a wound.

3. The bone screw structure is capable of lowering the area of the opening of the wound to recover the wound well.

4. The bone screw structure is capable of lowering the area of the opening of the wound to decrease an infection risk.

5. The bone screw structure will not break a patient's organization in surgery.

### BRIEF DESCRIPTION OF THE DRAWINGS

FIG. 1 is a perspective view showing the assembly of a bone screw structure for a minimally invasive surgery according to a preferred embodiment of the present invention.

FIG. 2 is a plan view showing the assembly of the bone screw structure for the minimally invasive surgery according to the preferred embodiment of the present invention.

FIG. 3 is a plan view showing the application of the bone screw structure for the minimally invasive surgery according to the preferred embodiment of the present invention.

FIG. 4 is a perspective view showing of a conventional bone screw structure.

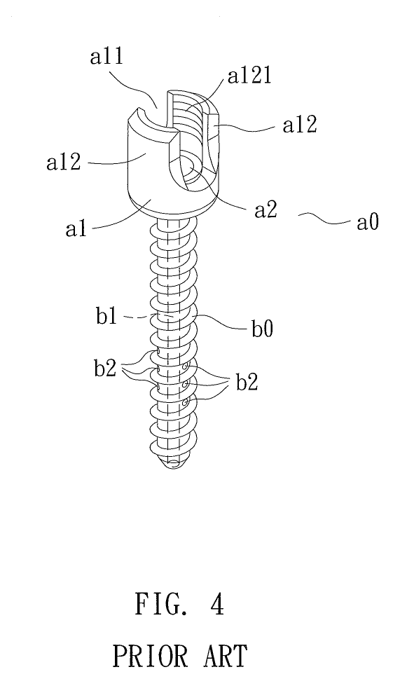

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

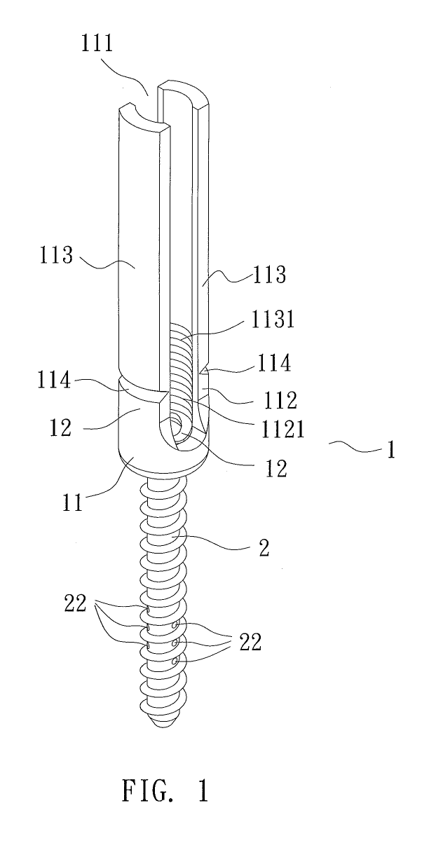

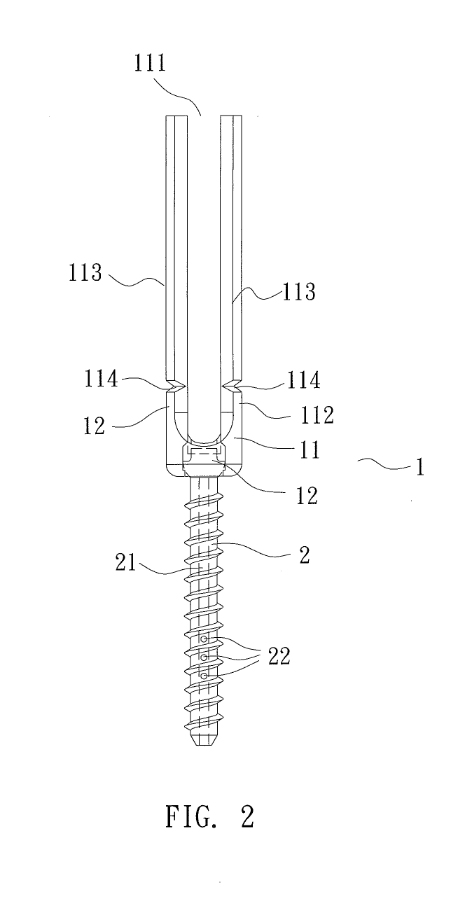

Referring to FIGS. 1 and 2, a bone screw structure for a minimally invasive surgery according to a preferred embodiment of the present invention comprises a body 1 and a screwing nail 2, wherein

the body 1 includes a holder 11 and an axial seat 12, the holder 11 has a through hole 111 and two first peripheral walls 112, and each first peripheral wall 112 has first threads 1121;

the screwing nail 2 is axially connected with the axial seat 12 of the body 1 and has an inner orifice 21;

characterized in that:

the each first peripheral wall 112 of the holder 11 includes a second side face 113 extending therefrom, between the each first peripheral wall 112 and the second side face 113 is defined a V-shaped slot 114, and the second side face 113 has second threads 1131.

The first threads 1121 and the second threads 1131 have an identical rotating direction and are formed successively.

The screwing nail 2 further includes a plurality of apertures 22 communicating with the inner orifice 21.

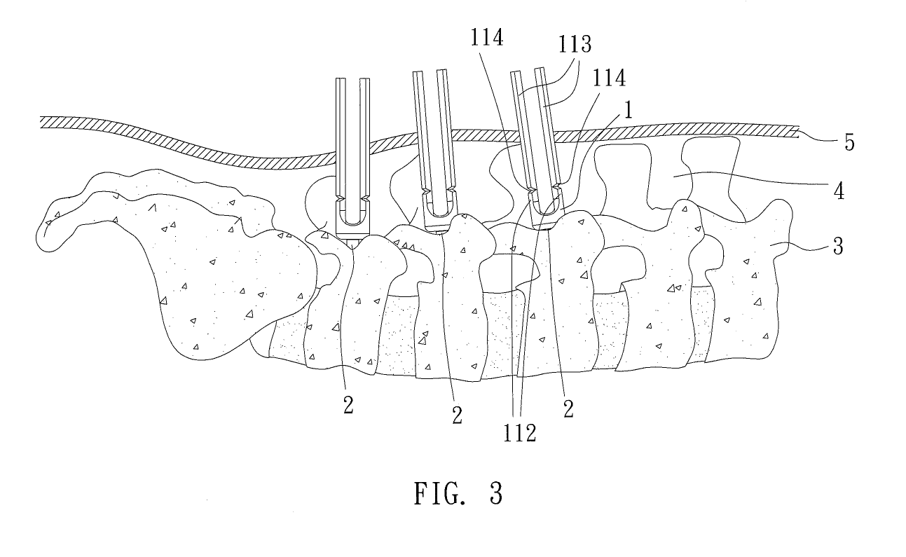

As shown in FIG. 3, in operation, the screwing nail 2 of the body 1 of the bone screw structure of the present invention is locked on a spine 3, and the second side face 113 of the each first peripheral wall 112 inserts through a skin 5 of a muscle tissue 4 to expose, i.e., as implanting the body 1 of a bone screw, a minimally invasive surgery is operated on a patient's body, and then the screwing nail 2 is screwed at a proper position of the spine 3. Because only a small opening is cut to form a small wound in the minimally invasive surgery, an infection risk is lowered and the patient recovers quickly. In addition, the V-shaped slot 114 between the each first peripheral wall 112 and the second side face 113 is provided to be a cut forcing portion of an auxiliary tool of the second side face 113 after implanting the bone screw, thus merely leaving the each first peripheral wall 112 of the body 1 in the patent's body.

Thereby, the bone screw structure of the present invention is used in the minimally invasive surgery, wherein the two first peripheral walls 112 of the holder 11 of the body 1 include two second side faces 113 extending therefrom, and between the two first peripheral wall 112 and the two second side faces 113 are defined two V-shaped slots 114 to provide an easy operating space to the bone screw and to lower an area of the opening of the wound.

While we have shown and described various embodiments in accordance with the present invention, it is clear to those skilled in the art that further embodiments may be made without departing from the scope of the present invention.

What is claimed is:

1. A bone screw structure for a minimally invasive surgery comprises:
a body including a holder and an axial seat, the holder having a through hole and two first peripheral walls, and each first peripheral wall having first threads;
the screwing nail being axially connected with the axial seat of the body and having an inner orifice;
characterized in that:
the each first peripheral wall of the holder including a second side face extending therefrom, between the each first peripheral wall and the second side face is defined a V-shaped slot, and the second side face has second threads.

2. The bone screw structure for the minimally invasive surgery as claimed in claim 1, wherein the first threads and the second threads have an identical rotating direction and are formed successively.

3. The bone screw structure for the minimally invasive surgery as claimed in claim 1, wherein the screwing nail includes a plurality of apertures communicating with the inner orifice.
